(19) United States
(12) Patent Application Publication
(10) Pub. No.: US 2009/0292334 A1
(43) Pub. Date: Nov. 26, 2009

(54) OPTIMIZING AND MONITORING ADAPTIVE CARDIAC RESYNCHRONIZATION THERAPY DEVICES

(76) Inventor: Rami Rom, Zichron Yaakov (IL)

Correspondence Address: YORAM TSIVION, P.O. BOX 1307, PARDES HANNA, 37111, IL

(21) Appl. No.: 11/721,449

(22) PCT Filed: Dec. 1, 2005
(86) PCT No.: PCT/IL05/01288

§ 371 Date: Jun. 12, 2007

(30) Foreign Application Priority Data
Dec. 12, 2006 (IL) 165729

(51) Int. Cl. A61N 1/08

(52) U.S. Cl. 607/27

(57) ABSTRACT

A system for remotely monitoring cardiac resynchronization therapy (CRT) devices and for optimizing location of implanted leads. The system displays a graph of the right ventricle pacing interval (PRV) vs. left ventricle pacing interval (PLV) diagram at maximal stroke volume and or a graph of a responder curve that demonstrates the stroke volume obtained beat after beat by the implanted hemodynamic sensor with dynamically optimized AV and VV parameters. The system lends itself easily to be used as a remote monitoring means for active and resting patients.

### FIELD OF THE INVENTION

The present invention relates generally to cardiac pacemaker and defibrillator devices and more specifically to methods for optimising cardiac resynchronization therapy devices.

### BACKGROUND OF THE INVENTION

Implanted pacemakers and intracardiac cardioverter defibrillators (ICD) deliver therapy to patients suffering from various heart-diseases (Clinical Cardiac Pacing and Defibrillation, 2^{nd} edition, Ellenbogen, Kay, Wilkoff, 2000). It is known that the cardiac output depends strongly on the left heart contraction in synchrony with the right heart (see U.S. Pat. No. 6,223,079). Congestive heart failure (CHF) is defined generally as the inability of the heart to deliver enough blood to meet the metabolic demand. Often CHF is caused by electrical conduction defects. The overall result is a reduced blood stroke volume from the left side of the heart. For CHF patients, a permanent pacemaker with electrodes in 3 chambers that are employed to re-synchronize the left and right ventricles contractions is an effective therapy, (“Device Therapy for Congestive Heart Failure”, K. Ellenbogen et al, Elsevier Inc. (USA), 2004). The resynchronization task demands exact pacing management of the heart chambers such that the overall stroke volume is maximized for a given heart rate (HR), where it is known that the main intent is to cause the left ventricle to contract in synchrony with the right ventricle. Clearly, the re-synchronization task is patient-dependent, and with each patient the best combination of pacing time intervals that restore synchrony are changed during the normal daily activities of the patient. For these reasons, next generation cardiac re-synchronization therapy devices are to have online adaptive capabilities in order to adjust to the hemodynamic performance.

The reasons that the currently available cardiac resynchronization therapy (CRT) devices cannot achieve optimal delivery of CRT are as follows:
1. Programming and troubleshooting CRT device—as of today, optimizing the CRT device using echocardiography is expensive, time consuming and operator dependent. The clinician is required to optimize both the atrioventricular delay (AV delay), and the interventricular delay (VV interval) in order to achieve resynchronization of heart chamber contractions.
2. Consistent Delivery of CRT—There are several reasons why CRT is not delivered consistently, and at times not delivered at all for hours. Two reasons for this are failure to optimise the AV delay and programming of the maximal tracking rate too low.
3. Follow ups—The clinician must perform the complex task of optimization and programming of the CRT device, first at the implantation and then at each follow-up.
4. CRT non-responders—a significant number of patients, about 30%, do not respond to CRT after implantation. The development of good markers that will enable identification of responders to CRT is a major issue due to the complexity of the instrumentation, the need for device implantation, and the medical costs associated with the treatment, (David A. Kaas, “Ventricular Resynchronization: Pathophysiology and Identification of Responders”, Reviews in Cardiovascular Medicine, Vol 4, Suppl2, 2003).

Hayes et al. in “Resynchronization and Defibrillation for Heart Failure, A Practical Approach”, Blackwell Publishing, 2004, suggest that optimal programming of the CRT device may turn “non responders” into “responders” and “responders” to better “responders”. The present invention provides a novel method for optimizing CRT devices which use the data obtained by dynamic active diagnostics, thus enabling a clinician to program the CRT device with the optimal AV and VV intervals obtained during an electrophysiology (EP) study and also to identify responders to CRT.

### BRIEF DESCRIPTION OF THE DRAWINGS

FIG. 1 is a graph showing the right ventricle pacing interval (PRV) vs. left ventricle pacing interval (PLV) graph with maximal stroke volume for three simulated patients I, II and III;

FIG. 2 is a graph showing the PRV vs. PLV plan with maximal stroke volume for three simulated patients IV, V and VI.

FIG. 3 is a graph comparing the simulated stroke volumes of a responder and a non-responder to CRT.

### DETAILED DESCRIPTION OF THE PRESENT INVENTION

The present invention provides an electrophysiological (EP) testing system, which enables the pacing of the ventricles, sensing the intracardiac electrograms and monitoring hemodynamic data in real time. An alternative application, is one in which the system of the invention employs an implanted biventricular pacemaker in which both AV delay and the VV interval are device parameters, programmed by a programmer or changed dynamically by an adaptive CRT and CRT-D (CRT device combined with a defibrillator) device, and the hemodynamic performance (such as the stroke volume) is monitored by an implanted sensor or by a non-invasive monitoring appliance.

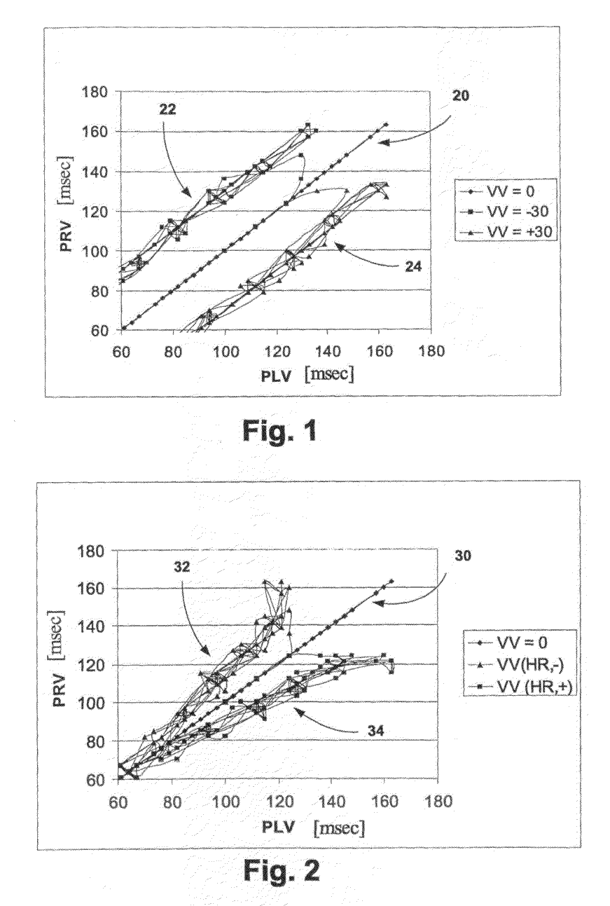

The present invention provides a method for dynamically diagnosing and optimising CRT (and CRT-D) devices or adaptive CRT (and CRT-D) devices, as described hereinbelow. For each heart rate, rest heart rate and at gradually higher heart rates, the pacing interval of the right and left ventricle are changed systematically. Accordingly, as indicated in FIG. 1 to which reference is made, the pacing intervals that produce the highest stroke volume at each heart rate are recorded and indicated in a vertical axis, referred to as PRV vertical axis (indicating the right ventricle pacing interval measured from the last atrial event). In the horizontal axis of the graph, referred to as PLV horizontal axis, the left ventricle pacing interval measured from the last atrial event are indicated.

The PRV vs. PLV diagram at maximal stroke volume shows the response of a patient to applied adaptive CRT effected continuously and at all heart rates and includes the information needed in order to optimally program CRT devices or adaptive CRT devices. The simulated results of three cases are shown. Graph 20 of simulated patent I receives simultaneous biventricular pacing at all heart rates, in which the highest stroke volume is obtained with a simultaneous pacing of both ventricles, i.e. VV interval=0, on the diagram diagonal with PRV=PLV. Graph 22 of simulated patent II has maximal stroke volumes when their left ventricle is paced 30 msec before the right ventricle continuously and at all heart rates, shown in the PRV vs. PLV diagram as a shifted curve to the upper part of the diagram above the diagonal line. Graph 24 of simulated patient III has maximal stroke volumes when the left ventricle is paced 30 msec after the right ventricle at all heart rates, shown in the PRV vs. PLV diagram as a shifted curve to the lower part of the diagram below the diagonal line.

FIG. 2 shows three results from three simulated cases. With these simulated patients, the simulated heart module left ventricle evoked response delay time was assumed to depend on the heart rate. At heart rate in rest, the left ventricle evoked response delay time parameter is maximal (+/−40 msec) and as the heart rate increases, the left ventricle evoked response delay time decreases. On the PRV vs. PLV diagram this dependency is seen clearly. In graph 30, simulated case IV is equivalent to the simultaneous CRT pacing shown in graph 20 of FIG. 1 to which reference is again made. Graph 32, case V in FIG. 2, is a simulated patient that needs left ventricle pacing earlier than right ventricle pacing and a variable VV interval. Graph 34 represents simulated patient VI, who needs the left ventricle pacing to lag behind the right ventricle with a variable VV interval.

Consequent to the above, the PRV vs. PLV diagram at maximal stroke volumes can be used as a dynamic diagnostic tool that presents graphically the characteristics of a heart failure patients response to CRT. It can be used to study the VV interval sign, magnitude and heart rate dependence all presented online in one diagram during a continuous electrophysiology study.

In a co-pending international patent application with the publication number WO 2005/007075, the contents of which are incorporated herein by reference, an adaptive CRT device is described (Implanted or an external device) in which the AV delays and the VV intervals are changed dynamically by the implanted device that hence performs dynamic optimization of these important pacing parameters (the AV delay and the VV interval). The change is effected in correspondence with the data derived from the hemodynamic sensor (invasive or non-invasive) in a closed loop using a neural network-learning module. With the adaptive CRT device, the PRV vs. PLV diagram of maximal stroke volume presented here is obtained automatically by the operating device and the diagram can be presented on a graphical interface, which is typically an electronic display device of an external programmer or on the external adaptive CRT device display screen.

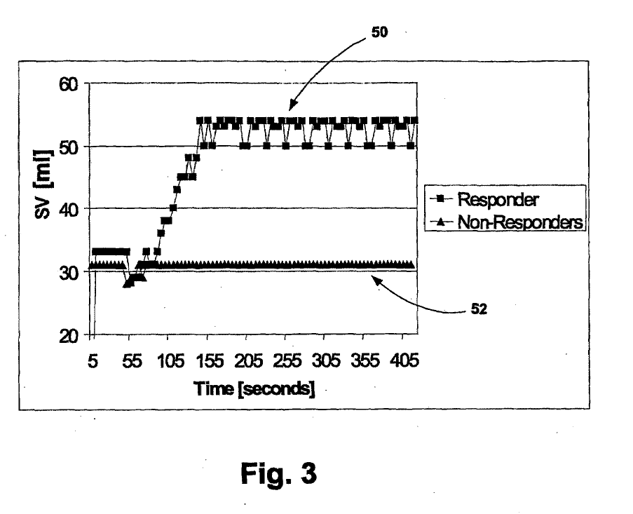

The adaptive CRT device (described in the above mentioned co-pending patent application) allows the identification of a responder to CRT during several minutes of continuous biventricular pacing in an electrophysiology test, or when programming an implanted adaptive CRT device. In FIG. 3 a comparison of the simulated stroke volumes in millilitres (ml) of a responder and a non-responder to CRT are described graphically as a function of time. At initialization the adaptive CRT device discerns the intrinsic conductance intervals of the patient. After a convergence criterion is met, an automatic switch to adaptive CRT mode occurs. In the adaptive CRT mode, the AV delay and VV interval are changed dynamically according to the information obtained from installed hemodynamic sensors. In the event that the patient is a responder to CRT, the device will change the pacing intervals in order to achieve a higher stroke volume as is depicted in graph 50. In the event that the patient is a non-responder, the stroke volume will not improve and will remain unchanged as seen in the graph 52. Upon identification of a non-responder, the clinician installing the CRT device may choose to change the implanted leads' positions in order to achieve an improved response to the CRT device.

Another aspect of the co-pending patent mentioned above is an external device to be used as an active diagnostic tool for optimization of implanted CRT devices. This can be used as a supplementary tool for a CRT device programmer. In accordance with the present invention, the PRV vs. PLV diagram at maximal stroke volumes represented by the responder curve, and the diagram as shown in FIG. 1 to which reference is again made, can be presented on a clinician's graphical interface (typically a display device) to enable the clinician to make a decision as regards the optimal AV delay and VV interval to be programmed in the implanted CRT device. With both external and implanted adaptive CRT devices, if the response to the CRT is not satisfactory, the clinician is able to change the lead position and restart the adaptive CRT device to repeatedly perform optimisations of the pacing interval until satisfactory results are obtained.

In addition to the active diagnostic benefit relating to implanted adaptive CRT devices explained above, which is typically implemented in a procedure room during the device implantation, the use of the PRV vs. PLV diagonal diagram and the responder curve diagram is beneficial in other aspects. It simplifies patients' follow-up routines at hospitals and clinics. It can also be transmitted using an RF communication channel from the implanted device at the patients home to a remote computer and hence to be used as a part of a remote telemedicine monitoring system. Such a monitoring system presents, according to this invention, the measured hemodynamic response to pacing with dynamically optimized AV and VV parameters beat after beat visually on external programmer screen or on a remote computer screen.

With regard to implanted adaptive CRT devices, in addition to monitoring the hemodynamic response to pacing with dynamically optimized AV and VV parameters as explained above, the PRV vs. PLV diagram and responder curve diagram can be used to monitor the pacing consistency and efficacy during various daily actives at rest and during exercise and hence can provide information otherwise unavailable today. The calculated stroke volume extracted from the hemodynamic sensor and the PRV vs. PLV diagram are two examples of analysis and presentation of the hemodynamic response to pacing therapy with dynamically optimized AV and VV parameters delivered by the implanted adaptive CRT device. The present invention is not limited only to these presentations of the adaptive CRT device operation, and any other such presentations of hemodynamic response to pacing with dynamically optimized AV and VV parameters are included in this invention.

AV delay optimization of dual chamber pacemakers and defibrillators are as important clinically as the AV delay optimization of CRT devices. Dual chamber devices use one atrial electrode and one ventricular electrode, and a ventricular pacing occurs after the pre-programmed AV delay measured from a sensed or paced atrial event ends. The AV delay depends on heart rate and on stress conditions and vary from patient to patient and during patients daily activities and therefore a fixed pre-programmed AV delay is a less then optimal solution. Loss of AV synchrony is a major cause for pacemaker syndrome as quoted in Beyerbach D. M. and Cadman C. Oct. 10, 2002, in http://www.emedicine.com/med/topic2919.htm“Pacemaker Syndrome”, the contents of which are incorporated herein by reference.

Ellenbogen et al. cited above, focused on clinical utility and proposed that “pacemaker syndrome represents the clinical consequences of AV dyssynchrony or sub-optimal AV synchrony, regardless of the pacing mode.”

The present invention for optimising and monitoring adaptive CRT (and CRT-D) devices is equally applicable to adaptive dual chamber devices with dynamic optimization of the AV delay only according to implantable hemodynamic sensor and using a neural network processor in the same way as performed with adaptive CRT device, described in the co-pending patent application WO 2005/007075.

1. A method for online diagnosis and for optimisation of a cardiac resynchronization therapy (CRT) device including hemodynamic sensors attached to the heart, comprising the steps of:
providing at least a graph of the right ventricle pacing interval (PRV) vs. left ventricle pacing interval (PLV) diagram at maximal stroke volume;
determining the optimal atrioventricular (AV) delay at all heart rates, and
determining the optimal (interventricular) VV interval at all heart rates.

2. A method for online diagnosis and for online diagnosis optimisation a cardiac resynchronization therapy (CRT) device as in claim 1, and wherein said CRT device employed for obtaining said VV and AV parameters is external.

3. A method for online diagnosis and for online diagnosis optimisation a cardiac resynchronization therapy (CRT) device as in claim 1, wherein positioning of the pacing leads, is made with reference to at least said graph.

4. A method for online diagnosing and for online diagnosis optimisation a cardiac resynchronization therapy (CRT) device as in claim 1, wherein positioning of the pacing leads is made with reference to a responder curve that demonstrates the stroke volume obtained beat after beat by the implanted hemodynamic sensor with dynamically optimized AV and VV parameters and presented on an external programmer graphical interface.

5. A method for online diagnosis and for online diagnosis optimisation a cardiac resynchronization therapy (CRT) device as in claim 1, wherein hemodynamic response to pacing with dynamically optimized AV and VV parameters and pacing consistency beat after beat in both rest and exercise is performed using a responder curve that presents the stroke volume obtained by the implanted hemodynamic sensor and/or the PRV vs. PLV diagonal diagram visually presented on a graphical interface of an external programmer during patients follow-up routines.

6. A method for remote monitoring of adaptive CRT device performance, hemodynamic response to pacing with dynamically optimized AV and VV parameters and pacing consistency beat after beat in both rest and exercise using a responder curve that presents the internally calculated stroke volume obtained by the implanted hemodynamic sensor and/or the diagonal diagram visually presented on a display device of an interface of a remote computer as part of a remote telemedicine monitoring system.

7. A diagnostic device for online optimization of a CRT device using at least one graph selected from the group consisting of PRV vs. PLV diagram at maximal stroke volume and a responder curve, said graphs visually presented on a graphical interface of an invasive electrophysiological testing system.

8. A diagnostic device for online optimization of an implanted CRT device as in claim 7 wherein said electrophysiological testing system is an implanted adaptive CRT device and said graphical interface is a display device of said adaptive CRT device programmer.

9. A system for a remote monitoring of an adaptive CRT device performance, for monitoring the patient's hemodynamic response to pacing with dynamically optimized AV and VV parameters and pacing consistency beat after beat in both rest and exercise, comprising:
a graphical interface of a remote computer;
at least a responder curve that presents the internally calculated stroke volume obtained by the implanted hemodynamic sensor, wherein said curve is displayed on said graphical interface, and
a means for communicating said internally calculated stroke volume to said remote computer.

10. A method for online diagnosing and for optimisation of an adaptive dual chamber pacemaker or defibrillator device including a hemodynamic sensor attached to the heart, comprising the steps of:
providing at least a responder curve that demonstrates the stroke volume obtained beat after beat;
finding the optimal atrioventricular (AV) delay at all heart rates.

11. A method for online diagnosis and for online diagnosis optimisation an adaptive dual chamber pacemaker or defibrillator device as in claim 10, wherein positioning of the pacing leads, is made with reference to a responder curve that demonstrates the stroke volume obtained beat after beat by the implanted hemodynamic sensor with dynamically optimized AV delay and presented on a display device of an external programmer's interface.

12. A method for online diagnosing and for online diagnosing optimisation an adaptive dual chamber pacemaker or defibrillator device as in claim 10, wherein hemodynamic response to pacing with dynamically optimized AV delay beat after beat in both rest and exercise is performed using a responder curve that presents the stroke volume obtained by the implanted hemodynamic sensor visually presented on graphical interface of an external programmer during patients follow-up routines.

13. A method for remote monitoring of an adaptive dual chamber pacemaker or defibrillator device performance, hemodynamic response to pacing with dynamically optimized AV delay beat after beat in both rest and exercise using a responder curve that presents the internally calculated stroke volume obtained by the implanted hemodynamic sensor visually presented in graphical interface of a remote computer as part of a remote telemedicine monitoring system.

14. A diagnostic device for online optimization of an adaptive dual chamber pacemaker or defibrillator device using a responder curve, said graph visually presented in graphical interface of an invasive electrophysiological testing system.

15. A diagnostic device for online optimization of an implanted adaptive dual chamber pacemaker or defibrillator device as in claim 14 wherein said electrophysiological testing system is an implanted adaptive dual chamber device and said graphical interface is a display device of a graphic interface of said adaptive dual chamber device programmer.

16. A system for remote monitoring of an adaptive dual chamber pacemaker or defibrillator device performance, for monitoring a patient's hemodynamic response to pacing with dynamically optimized AV delay beat after beat in both rest and exercise, comprising:
a graphical interface of a remote computer;
at least a responder curve that represents the internally calculated stroke volume obtained by the implanted hemodynamic sensor, wherein said curve is displayed on said graphical interface, and
a means for communicating said internally calculated stroke volume to said remote computer.
